(19) United States
(12) Patent Application Publication
(10) Pub. No.: US 2018/0361012 A1
(43) Pub. Date: Dec. 20, 2018

(54) HEMOSTATIC PRODUCTS

(71) Applicant: St. Teresa Medical, Inc., Eagan, MN (US)

(72) Inventors: Timothy Floyd, Eagle, ID (US); Philip A. Messina, Eagan, MN (US)

(21) Appl. No.: 16/013,510

(22) Filed: Jun. 20, 2018

Related U.S. Application Data
(60) Provisional application No. 62/522,288, filed on Jun. 20, 2017.

(51) Int. Cl. A61L 15/38; A61L 15/28; A61L 15/20

(52) U.S. Cl. CPC A61L 15/38; A61L 15/28; A61L 2400/04; A61L 2300/418; A61L 15/20

(57) ABSTRACT

A method of forming a hemostatic product. A solution is formed with a polymeric material. An electrospinning system is provided having a first nozzle and a second nozzle. The polymeric material solution is passed through the first nozzle and a hemostatic agent is passed through the second nozzle to form an electrospun fiber in which the polymeric material at least partially covers the hemostatic agent. The electrospun fiber is deposited on a support to form the hemostatic product.

### REFERENCE TO RELATED APPLICATION

This application claims priority to Provisional Applic. No. 62/522,288, filed on Jun. 20, 2017, the contents of which are incorporated herein by reference.

### FIELD OF THE INVENTION

The invention relates to products having hemostatic characteristics. More particularly, the invention relates to products having encapsulated hemostatic components.

### BACKGROUND OF THE INVENTION

The body's natural response to stem bleeding from a wound is to initiate blood clotting via a complex process known as the coagulation cascade. The cascade involves two pathways that ultimately lead to the production of the enzyme thrombin, which catalyzes the conversion of fibrinogen to fibrin.

Fibrin is then cross-linked to form a clot, resulting in hemostasis. For wounds that are not severe, and in individuals that have no countervening conditions, the body is usually able to carry out this process efficiently in a manner that prevents excessive loss of blood from the wound. However, in the case of severe wounds, or in individuals in whom the clotting mechanism is compromised, this may not be the case.

For such individuals, it is however possible to administer components of the coagulation cascade, especially thrombin and fibrinogen, directly to the wound to bring about hemostasis. Bandaging of bleeding wounds is also a usual practice, in part to isolate and protect the wounded area, and also to provide a means to exert pressure on the wound, which can also assist in controlling bleeding.

While these methods may be carried out satisfactorily in cases of mild trauma or under conditions of “controlled” wounding (e.g. surgery), many situations in which such treatments are most needed are also those in which it is the most difficult to provide them. Examples of such wounds include, for example, those inflicted during combat or unanticipated wounds that occur as the result of accidents. In such circumstances, survival of the wounded individual may depend on stopping blood loss from the wound and achieving hemostasis during the first few minutes after injury. Unfortunately, given the circumstances of such injuries, appropriate medical intervention may not be immediately available.

In particular, the treatment of penetrating wounds such as bullet wounds or some wounds from shrapnel is problematic. This is due to the difficulty in placing a hemostatic product and/or therapeutic agents at the actual site of injury, which includes an area that is well below the body surface and difficult or impossible to access using conventional techniques.

Jiang et al. in Biomacromolecules, v. 5, p. 326-333 (2004) teaches electrospun dextran fibers. Agents associated with the fibers (e.g. BSA, lysozyme) are directly electrospun into the fibers. The fibers may also include other polymers electrospun with the dextran.

Jiang et al. in Journal of Biomedical Materials Research Part B: Applied Biomaterials, p. 50-57 (2006) discloses electrospun fibers that are a composite of poly(c-caprolactone) as a shell and dextran as a core. These fibers provide the slow release of agents (bovine serum albumin, BSA) that are also electrospun into the fibers.

Smith et al., U.S. Pat. No. 6,753,454, discloses electrospun fibers comprising a substantially homogeneous mixture of a hydrophilic polymer and a polymer that is at least weakly hydrophobic, which may be used to form a bandage. The bandage may comprise active agents (e.g. dextran). However, the disclosed fibers are not readily soluble in liquid.

MacPhee et al., U.S. Pat. No. 6,762,336, teaches a hemostatic multilayer bandage that comprises a thrombin layer between two fibrinogen layers. The bandage may contain other resorbable materials such as glycolic acid or lactic acid based polymers or copolymers. Neither electrospun fibers nor dextran fibers are taught as components of the bandage.

Smith et al., U.S. Pat. No. 6,821,479, teaches a method of preserving a biological material in a dry protective matrix, the matrix comprising fibers such as electrospun fibers. One component of the fibers may be dextran, but homogeneous dextran fibers are not described.

Cochrum et al., U.S. Pat. No. 7,101,862, teaches hemostatic compositions and methods for controlling bleeding. The compositions comprise a cellulose containing article (e.g. gauze) to which a polysaccharide is covalently or ionically crosslinked. The crosslinked polysaccharide may be dextran. However, the compositions are not electrospun and exogenous clotting agents are not included in the compositions.

Wnek et al., U.S. Patent Publication No. 2004/0018226, discloses fibers produced by an electroprocessing technique such as electrospinning. The fibers comprise enclosures within the fibers for containing substances that are not miscible with the fibers. Dextran is not taught as a fiber component.

Fisher et al., U.S. Patent Publication No. 2007/0160653, teaches a hemostatic textile comprising hemostatic factors (e.g. thrombin, fibrinogen) but the fibers are formed from electrospun glass plus a secondary fiber (e.g. silk, ceramic, bamboo, jute, rayon, etc.)

Carpenter et al., U.S. Patent Publication No. 2008/0020015, teaches wound dressing comprised of various biodegradable polymers and hydrogels having allogenic or autologous precursor cells (e.g. stem cells) dispersed within the polymers. The polymers may be prepared by electrospinning, and one polymer component may be dextran. However, the polymers cannot be immediately soluble upon contact with liquid, as they must provide a scaffolding for delivery of the cells over time, even though the polymers eventually biodegrade in situ.

Li et al., U.S. Patent Publication No. 2008/0265469, describes electrospun nanofibers that may include dextran. However, the nanofibers are not described as readily soluble in liquids.

Eskridge et al., U.S. Patent Publication No. 2009/0053288, teaches a woven hemostatic fabric comprised of about 65% fiberglass yarn and about 35% bamboo yarn. The fiberglass component may be electrospun, and hemostatic factors such as thrombin may be associated with the fabric, e.g. by soaking the material in a solution of thrombin. This document indicates that dextran may be added as a hygroscopic agent.

There is an ongoing need to provide improved methods and means to initiate blood clotting in wounds to stop or at least slow blood loss. In particular, there is an ongoing need to improve the capability to readily promote hemostasis in severe wounds in a facile manner, especially under circumstances where immediate treatment by medical personnel is limited or unavailable.

Bowlin et al., U.S. Patent Publication No. 2011/0150973, discloses a method of delivering one or more agents of interest to a location of interest. The method includes applying or delivering to a location of interest a hemostatic product. The hemostatic product includes electrospun dextran fibers that dissolve upon contact with liquid. The hemostatic product also includes one or more agents of interest associated with said electrospun dextran fibers. Applying or delivering results in dissolution of the electrospun dextran fibers in liquid at the location of interest to thereby release the one or more agents of interest into the liquid.

### SUMMARY OF THE INVENTION

An embodiment of the invention is directed to a method of forming a hemostatic product. A solution is formed with a polymeric material. An electrospinning system is provided having a first nozzle and a second nozzle. The polymeric material solution passes through the first nozzle and a hemostatic agent passes through the second nozzle to form an electrospun fiber in which the polymeric material at least partially covers the hemostatic agent. The electrospun fiber is deposited on a support to form the hemostatic product.

Another embodiment is directed to a hemostatic product that includes an electrospun fiber. A polymeric material at least partially covers a hemostatic agent core.

Another embodiment of the invention is directed to method of forming a hemostatic product. A solution is formed with a polymeric material. A first electrospinning system is provided having a first nozzle and a second nozzle. The polymeric material solution is passed through the first nozzle on the first electrospinning system and a first hemostatic agent is passed through the second nozzle on the first electrospinning system to form a first electrospun fiber in which the polymeric material at least partially covers the first hemostatic agent. A second electrospinning system is provided having a first nozzle and a second nozzle. The polymeric material solution passes through the first nozzle on the second electrospinning system and a second hemostatic agent passes through the second nozzle on the second electrospinning system to form a second electrospun fiber in which the polymeric material at least partially covers the second hemostatic agent. The first hemostatic agent is different than the second hemostatic agent. The first electrospun fiber and the second electrospun fiber are deposited on a support to form the hemostatic product.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a schematic view of an electrospinning apparatus according to an embodiment of the invention.

FIG. 2 is a side view of an electrospinning nozzle for the electrospinning apparatus.

FIG. 3 is an end view of the electrospinning nozzle.

FIG. 4 is an end view of another embodiment of the electrospinning nozzle.

### DETAILED DESCRIPTION OF THE INVENTION

An embodiment of the invention is directed to a system for providing hemostasis in a person or animal. The system generally includes a hemostatic product that contains at least one hemostatic agent.

The hemostatic agent is encapsulated within the hemostatic product to reduce the potential of the hemostatic agent from becoming disassociated from the hemostatic product. Encapsulating the hemostatic agent also enhances the stability of the hemostatic product such that it is possible for the hemostatic product to be stored for an extended period of time prior to use without the hemostatic product experiencing degradation.

When the hemostatic product is applied to the injury site, the materials used to fabricate the hemostatic product dissolve to thereby release the active agents to the injury site and provide the hemostatic effect. The hemostatic product may be used in trauma situations where the condition of the patient must be stabilized until it is possible to transport the patient to a treatment facility having medical treatment equipment that is more advanced to the medical treatment equipment available where the patient was injured.

In some embodiments of the invention, only dextran and the hemostatic agents are used in the hemostatic product and thus after clot formation, there is no need to disturb the clot to remove hemostatic product components, since none remain at the site. The hemostatic product thereby does not leave any residual foreign bodies that elicit foreign body reactions or act as a nidus for infection. Furthermore, the hemostatic product does not contain any xenoproteins, which have the potential of eliciting immune reactions in persons on which the hemostatic product is used.

The components used in fabricating the hemostatic product should be selected to be the same as components found in a living body where the hemostatic product is to be used. Alternatively, the components used in fabricating the hemostatic product are compatible with and readily broken down when the hemostatic product is used on or in a living body.

Using such a process minimizes complications associated with components of the hemostatic product not being promptly being broken down as such a process could cause inflammation in the living body. The only thing that remains after the use of the hemostatic product is the clot, which most living bodies are adapted to degrade over time.

The hemostatic system generally includes a hemostatic product having a sheet that is fabricated from electrospun fibers in which the at least one hemostatic agent is encapsulated.

Electrospinning is a non-mechanical processing strategy and can be scaled to accommodate the large volumes necessary to meet the needs of commercial processing. Additional details on the electrospinning process are provided in U.S. application Ser. No. 12/937,322, the contents of which are incorporated herein by reference.

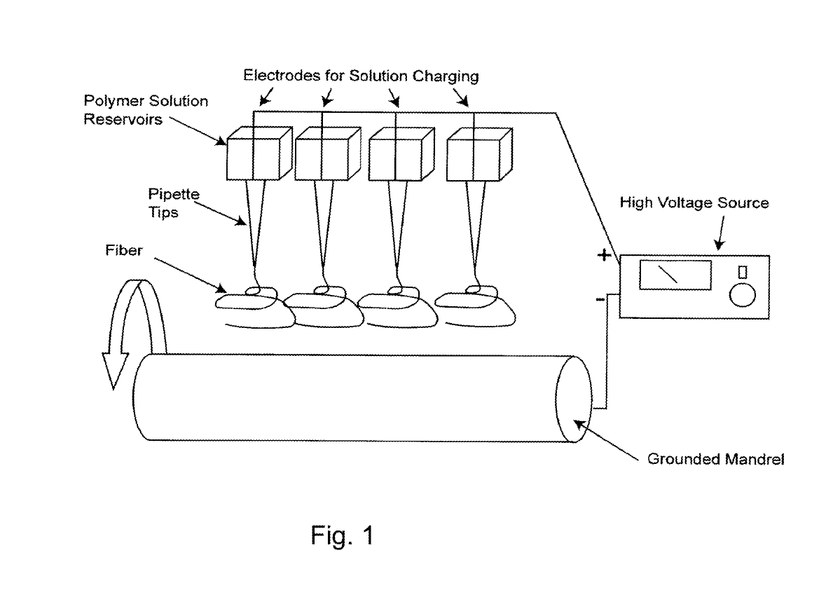

A schematic illustration of an electrospinning system used in conjunction with the electrospinning process is set forth in FIG. 1. A significant difference between this electrospinning system and the electrospinning system described in the application referenced above is that this system utilizes a multi-nozzle configuration.

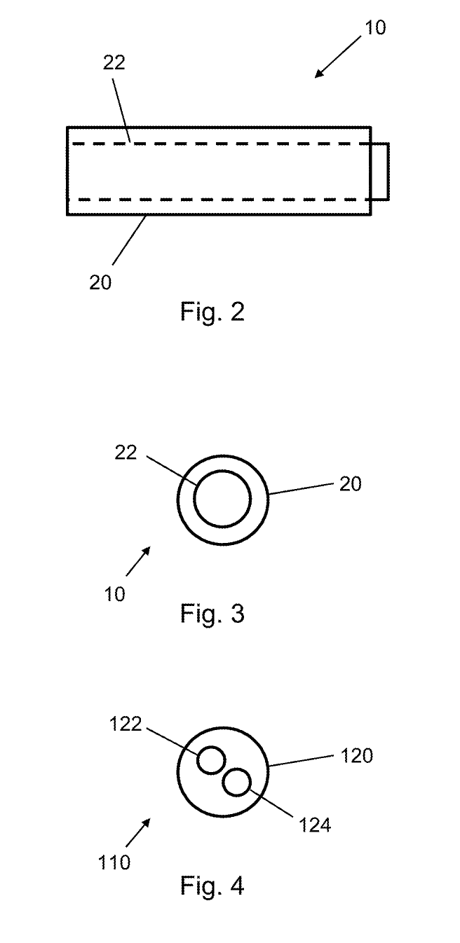

The multi-nozzle 10 includes a first nozzle 20 and a second nozzle 22. In certain embodiments, the first nozzle 20 extends at least partially around the second nozzle 22 as illustrated in FIGS. 2 and 3. In still other embodiments, the first nozzle 20 extends substantially around the second nozzle 22 as illustrated in FIG. 3. This configuration facilitates encapsulating the material fed through the second nozzle 22 with the material that is fed through the first nozzle 20.

The hemostatic agent may be fed through the second nozzle 22. In certain embodiments, the hemostatic agent is provided in a powder form that is propelled through the second nozzle 22 such as with a gas. In certain embodiments where the hemostatic agent includes thrombin and fibrinogen, the thrombin and fibrinogen may be mixed together before the hemostatic agent is fed through the second nozzle 22.

In other embodiments, the thrombin may be fed through a second nozzle 122 and the fibrinogen may be fed through a third nozzle 124 that are separate from each other but both located within the first nozzle 120 of the multi-nozzle device 110. Such a configuration may facilitate changing the rates at which the different hemostatic agents are added to the electrospun fibers. This configuration may also reduce the potential of interactions between the hemostatic agents prior to the hemostatic agents being incorporated into the electrospun fibers.

The concepts of the invention may also be expanded to use more than 3 nozzles when forming the electrospun fibers depending on the number of materials used in fabricating the electrospun fibers and the associated incompatibilities or desire to vary the rate at which the material is delivered.

In another embodiment, that includes multiple hemostatic agents, the hemostatic agents may be separately incorporated into the electrospun fibers using a separate electrospinning system for each of the hemostatic agents. As an alternative to using separate electrospinning systems, a single electrospinning system may be used with the individual hemostatic agents at separate times. In either of these situations, when forming the hemostatic product, layers having the different hemostatic agents will be placed in a stacked configuration.

In certain embodiments, the material fed through the first nozzle 20 is substantially dextran. As used herein, “substantially dextran” means that greater than about 90 percent of the material fed through the first nozzle 20 is dextran other than the solvent in which the dextran is dissolved. In other embodiments, the material fed through the first nozzle 20 is only dextran other than the solvent in which the dextran is dissolved.

The amount of dextran used in each hemostatic product can vary depending on the size of hemostatic product that is being manufactured, with typical hemostatic product formulations using from about 5-10 grams of dextran (usually 100,000-200,000 Mr) per hemostatic product.

Of more consequence is the concentration of dextran in the solution from which the fibers are electrospun. Generally, a solution of dextran for electrospinning will be of a concentration in the range of between about 0.1 and about 10 grams per milliliters of solvent. In other embodiments, the dextran concentration is between about 0.5 and about 5 grams per milliliter, and usually such a solution is at a concentration of about 1 gram per milliliter, which is about 0.15 milligrams. A preferred range would be from about 0.9 to about 1.1 grams of dextran per milliliter of solution that is to be electrospun.

Alternatively stated, the concentration of the dextran in the dextran and water solution is between about 40 percent by weight and about 60 percent by weight. In other embodiments, the concentration of the dextran is between about 45 percent by weight and about 55 percent by weight.

A diameter of the electrospun fibers may be affected such as by changing the rate at which the dextran solution is provided to the electrospinning machine. In certain embodiments, the electrospun fibers have a diameter of between about 3 micrometers and about 4 micrometers.

The area (length and width) of the hemostatic product of the invention can vary and be adjusted by adjusting spinning parameters. In addition, the mats of dextran fibers can be cut to a desired size after spinning. Generally, the hemostatic product will be from about 0.5 centimeters or less to about 30 centimeters or more in length and/or width, but larger or smaller sizes are also contemplated depending on the intended use of the hemostatic system.

Those of skill in the art will recognize that a variety of liquid solvents exist in which it is possible to dissolve dextran. However, superior results for electrospinning dextran are generally achieved when the solvent is water, especially deionized or distilled or deionized, distilled (ddH2O) or other forms of relatively pure water. In addition, there are no negative interactions during use of the hemostatic product associated with water remaining in the hemostatic product and there is far less environmental impact associated with the use of water as compared to many other solvents.

Usually the agents are bioactive agents that have a beneficial or therapeutic effect at the wound site. In one embodiment, the site is a bleeding wound at which it is desired to form a blood clot to stop or slow the bleeding. In this embodiment, the therapeutic substances of interest may include, for example, thrombin and fibrinogen, although other agents active in promoting hemostasis, including but not limited to capscian, may also be included.

The thrombin and/or fibrinogen that are used in the hemostatic product are in forms that are biologically active when they come into contact with blood. Hence upon dissolution, the thrombin acts on the fibrinogen, converting it to fibrin, which then forms a clot within the wound to thereby staunch the flow of blood.

In certain embodiments, the thrombin and fibrinogen may be derived from human sources. In other embodiments, the thrombin and fibrinogen are salmon thrombin and fibrinogen. Advantages of using salmon as a source of these materials include but are not limited to the lack of concern about transmission of etiologic agents (e.g. viruses) that may occur when human and other mammalian sources of thrombin or fibrinogen (e.g. bovine) are used.

The hemostatic agent is fed through the second nozzle 22 at a rate that is sufficient to provide the electrospun fibers with an effective concentration of the hemostatic agent for achieving hemostasis when the hemostatic product is applied to a wound. The effective concentration thereby depends on factors such as the amount of electrospun fibers that are used in the hemostatic product and the anticipated rate of blood flow that is intended to be stopped through the use of the hemostatic product.

The quantity of fibrinogen used in the hemostatic product may be adjusted by changing either the concentration of the fibrinogen in the hemostatic mixture or changing the rate at which the hemostatic mixture is used in the hemostatic product. The quantity of fibrinogen added to the hemostatic product is generally in the range of from about 10 milligrams to about 3 grams. In certain embodiments, the amount of fibrinogen in each of the hemostatic products is between about 20 milligrams to about 1 gram.

The quantity of thrombin used in the hemostatic product may be adjusted by changing either the concentration of the thrombin in the hemostatic mixture or changing the rate at which the hemostatic mixture is used in the hemostatic product. The quantity of thrombin added to each of the hemostatic products is generally between about 10 and 10,000 NIH Units. In certain embodiments, the amount of thrombin in each of the hemostatic products is between about 20 and 6,000 NIH Units.

In addition to the hemostatic agents used in the electrospun fibers, active agents may also be associated with the electrospun dextran base using a variety of techniques that are known to those of skill in the art, and will depend in part on the precise form of the substance and the means at hand. For example, for powdered, particulate thrombin and fibrinogen, association may be carried out by sprinkling, shaking, blowing, etc. the agents onto a layer of the excipient or carrier.

In some embodiments, active agents such as thrombin may be electrosprayed with sucrose to form sugar droplets, which tends to stabilize thrombin and can also “trap” other substances of interest for delivery to the hemostatic product. In other embodiments, the therapeutic agents may themselves be electrospun. For example, the therapeutic agents are dissolved in and electrospun from a solution. The active agents may be electrospun into other forms such as droplets, beads, etc.

In addition, electrospun or non-electrospun collagen, agents that absorb water, various dry salts that would tend to absorb fluids when placed in contact with e.g. blood; engineered thrombin or thrombin mimics; engineered fibrinogen; agents that cause vasospasm (e.g. ADP, 5-hydroxytryptamine, 5-HT and thromboxane, (TXA-2) to help contract and seal a bleeding vessel, etc. may also be included.

Other components of the clotting cascade may be added to the hemostatic product, for example: tissue factors that are normally only expressed on the surface of damaged cells and that start the normal clotting cascade; serotonin which enhances platelet clumping and promotes vessel constriction; and other agents that are used to replace missing components of the clotting cascade in hemophilia, for example, factor 7 (which activates the so called external extrinsic coagulation cascade) and crude extracts of platelets.

Active agents that function to promote late stages of wound healing may also be included to, for example, facilitate cell migration and remodeling. The incorporation of collagen is an example of such an active agent.

The therapeutic agents must be amenable to drying and are associated with the other components of the hemostatic product in the dry state, since liquid may negatively affect at least one of the components used in the hemostatic product. For example, the active agents may be desiccated or lyophilized, or water may be removed by other means.

In certain embodiments, the material is electrospun onto a vacuum table to retain the electrospun material in a substantially stationary position during the fabrication process. Retaining the electrospun material in the substantially stationary position enhances the ability to fabricate the hemostatic product such as placing components on the electrospun material and accurately cutting the electrospun material into the hemostatic product.

It is possible for the hemostatic product to be formed in more than one layer. Using such a process enhances the ability to control the amount of hemostatic materials that are used because multiple layers may be incorporated into the hemostatic product. A hemostatic product may contain 1-2 layers. In other embodiments the hemostatic product may include between 2-20 layers.

The height or thickness of the hemostatic product can vary considerably depending on the intended use of the hemostatic product. In certain embodiments, the hemostatic product has a thickness of between about 0.1 millimeter and about 5 centimeters. In other embodiments, the thickness of the hemostatic product is between about 0.3 millimeters and about 0.50 millimeters.

The thickness of the hemostatic product (which is related to the volume) may impact the rate of dissolution of the dextran upon contact with liquid. For example, a thin hemostatic product (e.g. about 2 millimeters) will dissolve more rapidly than a hemostatic product that is thicker, providing the loft of the fibers is comparable.

In most embodiments, dissolution of the dextran fibers is extremely rapid, e.g. about 5 minutes or less after exposure to liquid, or about 4 minutes or less, or about 3 minutes or less, or about 2 minutes or less, or about 1 minute or less. In certain embodiments, the hemostatic product substantially dissolves in between about 1 second and about 20 seconds. As used herein, “substantially dissolves” means that less than about 20 percent of the electrospun fibers remain in fiber form. In other embodiments, “substantially dissolves” means that less than about 10 percent of the electrospun fibers remain in fiber form.

This rapid dissolution may be referred to herein as “instantaneous” or “immediate” dissolution. Compression of an electrospun dextran mat may be used to modulate the rate of dissolution, with greater levels of compression inversely impacting the rate, i.e. generally, the greater the degree of compression, the slower the rate of dissolution.

The rapid rate of dissolution is advantageous, particularly when delivering biologically active agents (e.g. hemostatic agents) to a site of action such as a wound. Rapid dissolution of the carrier dextran fibers provides extremely rapid delivery of the hemostatic agents to the wound upon deployment of the hemostatic product.

If thrombin is included in the hemostatic product, it may be desirable to reduce the moisture content of the hemostatic product (e.g. a bandage or gauze) to less than about 5% to preserve thrombin activity during sterilization. This moisture content reduction can be achieved by drying the fabricated hemostatic product, e.g., under a vacuum, or by using a fabrication method that reduces moisture content from the beginning.

To minimize the potential of degradation of the hemostatic product, the hemostatic product should be protected from exposure to moisture because when the components used in the hemostatic product are exposed to moisture, the components degrade such as by dissolving.

The hemostatic product may include one or more stabilizers such as is described in U.S. application Ser. No. 13/622,690, which is assigned to the assignee of the present application and the contents of which are incorporated herein by reference. The stabilizers may enhance the ability of the hemostatic product to dissolve when the hemostatic products are applied to the injury site.

Prior to use of the hemostatic product, it may be desirable for the hemostatic product to be carried by a person on whom the hemostatic product could potentially be used and/or by a person who could potentially use the hemostatic product. In other embodiments, the hemostatic product resists degradation at temperatures of more than 140° F. to less than 0° F.

In certain embodiments, the hemostatic product should resist degradation when exposed to the elevated temperature such as up to about 150° F. for more than about 3 hours. In other embodiments, the hemostatic product should resist degradation when exposed to the elevated temperature for up to about 24 hours.

A threshold for the hemostatic product to be viewed as not experiencing degradation is that the hemostatic product does not exhibit noticeable visible physical changes when viewing the hemostatic product without magnification. The hemostatic product should also not experience noticeable physical changes when the hemostatic product is examined with magnification such as with a magnifying glass or a microscope.

The preceding characteristics should be displayed by the hemostatic product regardless of whether the hemostatic product is retained in the packaging materials while exposed to the elevated temperature conditions.

The stabilizer also enhances the usable shelf life of the hemostatic product. In certain embodiments, the stabilizer provides the hemostatic product with a shelf life of at least about 2 years. In other embodiments, the hemostatic product exhibits a shelf life of at least 3 years. As used herein, the term usable shelf life means that the hemostatic product does not exhibit noticeable degradation when viewed without magnification or with magnification such as a magnifying glass or microscope.

In some embodiments of the invention, the hemostatic products also include one or more support structures or support materials incorporated therein. For example, a backing may be incorporated into the hemostatic product.

The support material may be formed from various electrospun materials such as polyglycolic acid (PGA), polylactic acid (PLA), and their copolymers (PLGAs); charged nylon, etc. In one embodiment, the support material is compressed electrospun dextran fibers. By “compressed electrospun dextran fibers,” it is meant that electrospun dextran fibers are compressed together under pressure.

The support material may or may not be soluble in liquid, or may be slowly soluble in liquid, and may or may not be permeable to liquid. Slowly soluble materials include those from which absorbable or dissolving (biodegradable) stitches or sutures are formed, included PGA, polylactic and caprolactone polymers.

In certain embodiments, the support material may dissolve relatively quickly such as less than about 1 hour. In other embodiments, the support material may dissolve within from about 10 days to 8 weeks. In either case, the support material provides the advantage of not having to remove the hemostatic product and risk disrupting the clot.

However, in any case, the support material should not interfere with the immediate dissolution of the hemostatic product and delivery of the active agents associated therewith into the liquid that dissolves the hemostatic product.

All such arrangements, shapes, and embodiments of carrier layers and support materials as described herein are intended to be encompassed by the invention.

The hemostatic product may be sterilized prior to use, generally by using electromagnetic radiation, for example, X-rays, gamma rays, ultraviolet light, etc. Typically, the hemostatic products are sterilized using X-rays in a dose of about 5 kilograys (kGray). Any method that does not destroy the carrier or the activity of substances associated with the fibers may be used to sterilize the hemostatic products of the invention.

The hemostatic product may also include diagnostic agents that can be used by the treating medical professional to diagnose the nature of the injury. In certain embodiments, the diagnostic agent may change colors to indicate the presence of particular chemicals in the blood or to indicate particular characteristics of the blood. For example, if the patient is currently taking medications that cause thinning of the patient's blood. The diagnostic agents could also change colors to indicate the oxygen and/or glucose level of the blood.

In other embodiments, the products of the invention need not comprise agents that promote clotting. Those of skill in the art will recognize that the products of the invention are highly suitable for delivering many substances of interest to a desired liquid environment or location. For example, the products may be designed for delivery of therapeutic or beneficial substances to any moist environment of the body, where there is sufficient liquid to dissolve the electrospun dextran fibers and release the active substance, and where dissolved dextran is not problematic.

Such substances may include, for example, enzymes or their precursors (e.g. pro-enzymes or zymogens) and their substrates, substances that activate a protein or enzyme (e.g. proteases, cofactors, etc.), and the like.

For example, hemostatic products comprised of only thrombin might be used for small injuries or in combination with other interventions. In addition, other therapeutically beneficial substances may also be associated with the hemostatic product, including but not limited to: antibiotics, antiviral agents, anti-helminthic agents, anti-fungal agents, medicaments that alleviate pain, growth factors, bone morphogenic protein, vasoactive materials (e.g. substances that cause vasospasms), steroids to reduce inflammation, chemotherapy agents, contraceptives, etc.

Examples include but are not limited to oral, nasal, tracheal, anal, lung, and vaginal delivery of substances such as anti-microbial agents, analgesic agents, nutritional agents, etc. Oral applications include the delivery of substances useful for dental treatments, e.g. antibiotics, pain medications, whitening agents, etc.

In some embodiments, no bodily fluid is present (or if insufficient body fluid is present) and the applied hemostatic product can be “activated” by wetting, e.g. by spraying, or by otherwise applying a source of moisture (e.g. by exposing the hemostatic product to a moist material such as a sponge), or dropping hemostatic products into a liquid (e.g. a body of water), to cause release of the agents of interest associated with the dextran fibers.

The electrospun dextran fiber hemostatic products of the invention may serve as a “scaffolding” or carrier for containing, storing and/or transporting the substance(s) until use, i.e. until contacted with liquid that dissolves the electrospun dextran fibers, concomitantly releasing the substances into the liquid. Such substances may include, for example, enzymes or their precursors (e.g. pro-enzymes or zymogens) and their substrates, substances that activate a protein or enzyme (e.g. proteases, cofactors, etc.), and the like.

One of the challenges in successfully treating a wound, especially a wound where there is significant blood flow, is to achieve hemostasis. In addition to applying a hemostatic product such as is described in the other portions of this patent application, pressure is applied to the wound to enhance the likelihood that hemostasis will be achieved.

In certain embodiments, the pressure is provided by direct manual pressure such as using a human hand. In other embodiments, a material is placed over the wound and the direct manual pressure is used to hold the material in place.

The material may have absorbent capabilities such that blood and other fluids that are in proximity to the material are absorbed into the material. In such situations, it is possible for the clot to become associated with the material such as on the surface of the material or at least partially in the matrix of the material.

In addition to being used to produce hemostasis in humans, the concepts of the invention may be adapted for use in conjunction with other animals. Examples of such animals on which the invention can be used include dogs and cats.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A method of forming a hemostatic product comprising:
forming a solution with a polymeric material;
providing an electrospinning system having a first nozzle and a second nozzle;
passing the polymeric material solution through the first nozzle and passing a hemostatic agent through the second nozzle to form an electrospun fiber in which the polymeric material at least partially covers the hemostatic agent; and
depositing the electrospun fiber on a support to form the hemostatic product.

2. The method of claim 1, wherein the polymeric material substantially covers the hemostatic agent.

3. The method of claim 1, wherein the first nozzle extends at least partially around the second nozzle.

4. The method of claim 1, wherein the polymeric material is dextran.

5. The method of claim 1, wherein the hemostatic agent comprises at least one of fibrinogen and thrombin.

6. The method of claim 1, wherein the hemostatic agent is provided in a powder form.

7. The method of claim 1, and further comprising encapsulating the hemostatic agent in a stabilizing agent, wherein the stabilizing agent comprises sucrose.

8. A hemostatic product comprising an electrospun fiber wherein a polymeric material at least partially covers a hemostatic agent core.

9. The hemostatic product of claim 8, wherein the polymeric material substantially covers the hemostatic agent core.

10. The hemostatic product of claim 8, wherein the polymeric material comprises dextran.

11. The hemostatic product of claim 8, wherein the hemostatic agent at least one of fibrinogen and thrombin.

12. The hemostatic product of claim 8, wherein the hemostatic agent is in a powder form.

13. The hemostatic product of claim 8, wherein the hemostatic agent is encapsulated in a stabilizing agent.

14. The hemostatic product of claim 13, wherein the stabilizing agent comprises sucrose.

15. The hemostatic product of claim 8, wherein the polymeric material stabilizes the hemostatic agent core.

16. A method of forming a hemostatic product comprising:
forming a solution with a polymeric material;
providing a first electrospinning system having a first nozzle and a second nozzle;
passing the polymeric material solution through the first nozzle on the first electrospinning system and passing a first hemostatic agent through the second nozzle on the first electrospinning system to form a first electrospun fiber in which the polymeric material at least partially covers the first hemostatic agent;
providing a second electrospinning system having a first nozzle and a second nozzle;
passing the polymeric material solution through the first nozzle on the second electrospinning system and passing a second hemostatic agent through the second nozzle on the second electrospinning system to form a second electrospun fiber in which the polymeric material at least partially covers the second hemostatic agent, wherein the first hemostatic agent is different than the second hemostatic agent; and
depositing the first electrospun fiber and the second electrospun fiber on a support to form the hemostatic product.

17. The method of claim 16, wherein the polymeric material substantially covers the first hemostatic agent in the first electrospun fiber and the polymeric substantially covers the second hemostatic agent in the second electrospun fiber.

18. The method of claim 16, wherein the first nozzle extends at least partially around the second nozzle in the first electrospinning system and the second electrospinning system.

19. The method of claim 16, wherein the polymeric material is dextran and wherein the first hemostatic agent comprises fibrinogen and the second hemostatic agent comprises thrombin.

20. The method of claim 16, wherein the first hemostatic agent and the second hemostatic agent are provided in a powder form.

21. The method of claim 16, and further comprising encapsulating at least one of the first hemostatic agent and the second hemostatic agent in a stabilizing agent, wherein the stabilizing agent comprises sucrose.
